(19) United States
(12) Patent Application Publication
(10) Pub. No.: US 2025/0302432 A1
(43) Pub. Date: Oct. 2, 2025

(54) ULTRASOUND PROBE

(71) Applicant: FUJIFILM Corporation, Tokyo (JP)

(72) Inventor: Koichi ISHIDA, Tokyo (JP)

(73) Assignee: FUJIFILM Corporation, Tokyo (JP)

(21) Appl. No.: 19/095,444

(22) Filed: Mar. 31, 2025

(30) Foreign Application Priority Data
Apr. 1, 2024 (JP) 2024-059048

(51) Int. Cl. A61B 8/12; A61B 8/00; A61B 90/00

(52) U.S. Cl. CPC A61B 8/12; A61B 8/4444; A61B 90/39; A61B 2090/3937

(57) ABSTRACT

An ultrasound probe inserted into a body cavity includes: an ultrasound transducer that radiates an ultrasound wave; and a case that has a substantially cylindrical shape and that accommodates the ultrasound transducer. The case includes a direction marker that is a surface indicating a radiation direction of the ultrasound wave. The direction marker is provided at a position where the direction marker is visible from an outside and at least a portion of the direction marker overlaps a radiation range of the ultrasound wave in an axial direction.

### CROSS REFERENCE TO RELATED APPLICATION

This application claims priority to Japanese Patent Application No. 2024-059048 filed on Apr. 1, 2024, which is incorporated herein by reference in their entireties including the specifications, claims, drawings, and abstracts.

### BACKGROUND OF THE INVENTION

### 1. Field of the Invention

The present specification discloses an ultrasound probe that is inserted into a body cavity.

### 2. Description of the Related Art

In the related art, a technique has been known in which an ultrasound probe is inserted into a body cavity and ultrasound diagnosis is performed in order to check an internal state of an organ of a subject (for example, WO2015/166302A and the like). In the ultrasound diagnosis, the ultrasound probe is brought into contact with a surface of the organ. In this state, ultrasound waves are transmitted from the ultrasound probe to the inside of the organ, and reflected waves thereof are received. Then, an ultrasound tomographic image indicating the internal state of the organ is formed based on the obtained reflected wave signal. An operator, such as a doctor, performs various treatments, for example, collection of cells or injection of a drug by a puncture needle on the inside of the organ, while referring to the obtained ultrasound tomographic image.

Here, there is a demand for easily and clearly knowing an accurate correspondence relationship between the ultrasound tomographic image and the position of the actual organ. For example, the operator wants to know from which tomographic plane of the actual organ the ultrasound tomographic image has been obtained.

### SUMMARY OF THE INVENTION

WO2015/166302A discloses an ultrasound probe that is inserted into the body cavity. The ultrasound probe disclosed in WO2015/166302A has a substantially cylindrical shape so as not to damage biological tissue.

The problem with the substantially cylindrical ultrasound probe is that, although invasiveness to a living body is reduced, it is difficult to know a rotation angle of the ultrasound probe about an axis and thus a radiation direction of the ultrasound waves. In a case where the radiation direction of the ultrasound waves is not accurate, it is not possible to accurately discriminate from which tomographic plane of the actual organ the ultrasound tomographic image has been obtained. The ultrasound probe disclosed in WO2015/166302A is not capable of solving this problem.

Therefore, the present specification discloses an ultrasound probe that enables a user to easily discriminate a radiation direction of an ultrasound wave.

According to an aspect of the present invention, there is provided an ultrasound probe that is inserted into a body cavity and that comprises: an ultrasound transducer that radiates an ultrasound wave; and a case that has a substantially cylindrical shape and that accommodates the ultrasound transducer. The case includes a direction marker that is a surface indicating a radiation direction of the ultrasound wave. The direction marker is provided at a position where the direction marker is visible from an outside and at least a portion of the direction marker overlaps a radiation range of the ultrasound wave in an axial direction.

The provision of the direction marker enables an operator to easily discriminate the radiation direction of the ultrasound wave. In addition, the direction marker is disposed at the position where at least a portion of the direction marker overlaps the range of the radiation range of the ultrasound wave in the axial direction. The radiation range of the ultrasound wave is a range that the operator particularly pays attention to. The disposition of the direction marker in the vicinity of the radiation range makes it possible to suppress the movement of the line of sight of the operator to a small extent and makes it possible for the operator to observe the area around the radiation range more carefully.

In this case, a range of the direction marker in the axial direction may be the same as a range of the radiation range of the ultrasound wave in the axial direction, and the direction marker may also function as a range marker that indicates the radiation range of the ultrasound wave.

This configuration enables the operator to observe the direction marker to know both the radiation range and the radiation direction of the ultrasound wave at the same time.

In addition, the direction marker may be a surface that is parallel to the radiation direction of the ultrasound wave or a surface that is orthogonal to the radiation direction of the ultrasound wave.

This configuration enables the operator to easily and clearly know the radiation direction of the ultrasound wave from the inclination of the surface.

Further, the direction marker may be a substantially rectangular plane that is provided on a side opposite to a radiation surface of the ultrasound wave in a circumferential direction.

Since the direction marker has a rectangular shape, the operator can easily determine the inclination of the direction marker and thus the radiation direction of the ultrasound wave from the appearance of the angle of the side or corner portion of the rectangle.

Furthermore, the case may have a recessed portion that is recessed from a surrounding area, and the direction marker may be disposed in the recessed portion.

This configuration makes it possible to effectively prevent interference between the direction marker and a facing tissue.

Moreover, the case may further include one or more range markers that are provided at positions different from a position of the direction marker and that indicate the radiation range of the ultrasound wave.

This configuration makes it possible to check the radiation range of the ultrasound wave from various directions. As a result, even in a case where a visual field is limited as in an endoscope, the operator can accurately know the radiation range of the ultrasound wave.

In addition, a color of the direction marker may be opposite to a color of the case.

This configuration enables the operator to clearly identify the range marker.

According to the ultrasound probe disclosed in the present specification, it is possible to easily discriminate the radiation direction of the ultrasound wave.

### BRIEF DESCRIPTION OF THE DRAWINGS

FIG. 1 is a schematic view showing an aspect of use of an ultrasound probe.

FIG. 2 is a perspective view showing a distal end portion of the ultrasound probe.

FIG. 3 is a side view showing the distal end portion of the ultrasound probe and shows an ultrasound tomographic image.

FIG. 4 is a cross-sectional view taken along line A-A of FIG. 3.

FIG. 5 is a cross-sectional view taken along line B-B of FIG. 4.

FIG. 6 is an image diagram showing a relationship between a radiation direction of an ultrasound wave and a target part.

FIG. 7 is a view showing an example of another ultrasound probe.

FIG. 8 is a view showing an example of another ultrasound probe.

### DESCRIPTION OF THE PREFERRED EMBODIMENTS

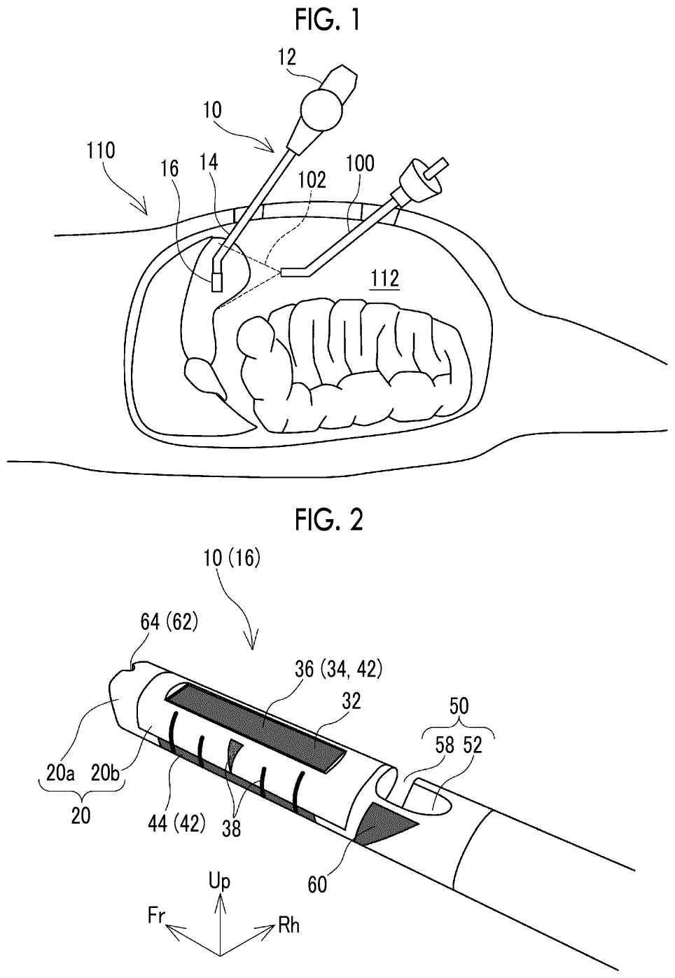

Hereinafter, a configuration of an ultrasound probe 10 will be described with reference to the drawings. FIG. 1 is a schematic view showing an aspect of use of the ultrasound probe 10. In addition, hereinafter, a laparo-probe used in an abdominal cavity will be described as an example. However, the technology disclosed in the present specification is not limited to the laparo-probe as long as it is inserted into a subject 110, and other types of ultrasound probes 10 may be used. In addition, the subject may be a person or an animal.

The ultrasound probe 10 according to the present example is used in laparoscopic surgery. In the laparoscopic surgery, an operator inserts an endoscope 100, the ultrasound probe 10, and other surgical instruments (for example, forceps, an electric scalpel, and the like) (not shown) from a port into an abdominal cavity 112. A camera is provided in the endoscope 100, and a video captured by the camera is displayed on a display (not shown) in real time. The operator operates the ultrasound probe 10 or other surgical instruments while observing the video on the display.

In addition, the operator knows an internal state of an organ (for example, a liver) in a body cavity using the ultrasound probe 10. Then, the operator performs a predetermined treatment (for example, tumor resection or the like) on the organ based on the obtained information.

The ultrasound probe 10 is roughly divided into an operation portion 12, an insertion portion 14, and a distal end portion 16. The operation portion 12 is a portion held by the operator. The operation portion 12 is provided with a plurality of controls (for example, buttons, dials, and the like) that receive various operations.

The insertion portion 14 is a tubular member that is inserted into the subject. The insertion portion 14 can be bent by operating the operation portion 12, and the position and orientation of the distal end portion 16 are changed by bending the insertion portion 14. A signal cable for transmitting and receiving an electric signal and a transmission cable for transmitting a force for bending the insertion portion 14 are provided in the insertion portion 14.

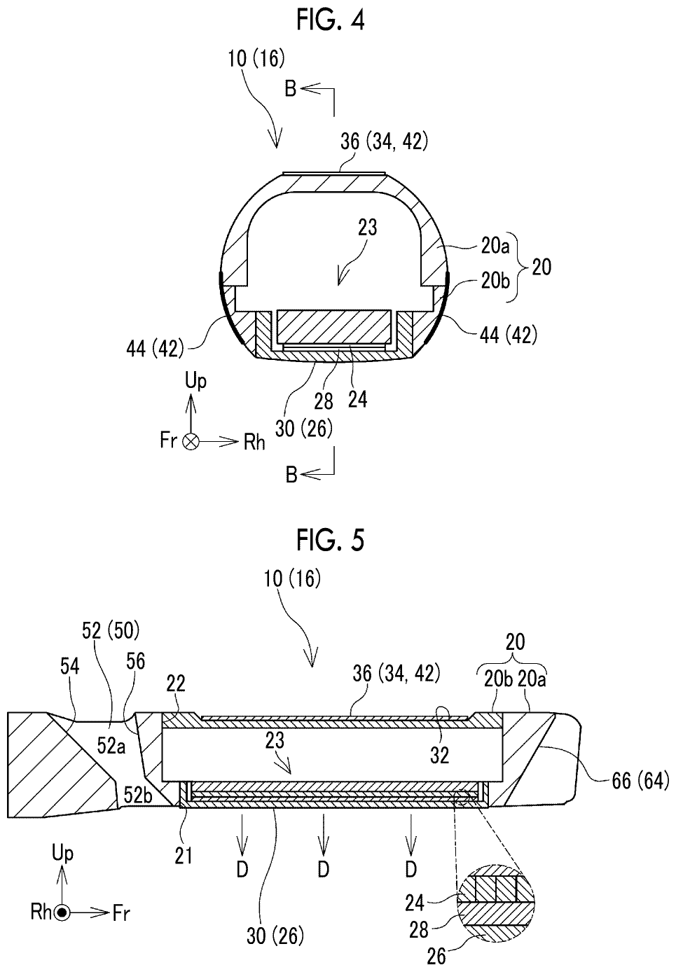

The distal end portion 16 is attached to a terminal of the insertion portion 14. The distal end portion 16 has an ultrasound transducer 23 (not shown in FIG. 1, see FIGS. 4 and 5) and transmits and receives ultrasound waves. The distal end portion 16 will be described with reference to FIGS. 2 to 5.

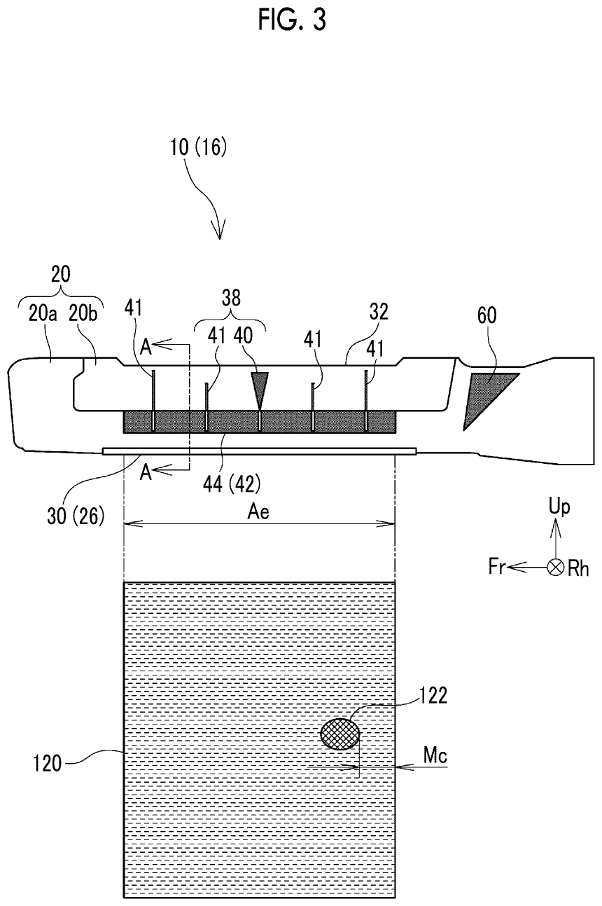

FIG. 2 is a perspective view showing the distal end portion 16. In addition, FIG. 3 is a side view showing the distal end portion 16 and is an image diagram showing an ultrasound tomographic image 120 obtained by the ultrasound probe 10. Further, FIG. 4 is a cross-sectional view taken along line A-A of FIG. 3, and FIG. 5 is a cross-sectional view taken along line B-B of FIG. 4. Furthermore, hereinafter, in order to clarify directions, an axial direction of a case 20 is referred to as a “front-rear direction”, a direction parallel to a radiation direction D of the ultrasound waves is referred to as an “up-down direction”, and a direction orthogonal to the front-rear direction and the up-down direction is defined as a “left-right direction”.

The distal end portion 16 has the case 20 that has a substantially cylindrical shape and the ultrasound transducer 23 that is accommodated in the case 20. The ultrasound transducer 23 has a plurality of transducer elements 24 (see FIG. 5) that transmit and receive the ultrasound waves. Further, in FIG. 5, the transducer element 24 is shown to be larger or smaller than the actual size for ease of understanding. The ultrasound probe 10 according to the present example is a linear probe that performs linear scanning with an ultrasound beam, and the plurality of transducer elements 24 are linearly arranged in the front-rear direction (that is, the axial direction of the case 20). A matching layer 28 and an acoustic lens 26 are disposed in a thickness direction of the transducer element 24. The ultrasound beam passes through the matching layer 28 and the acoustic lens 26 and is radiated to the outside of the ultrasound probe 10. Therefore, an outer surface of the acoustic lens 26 is a radiation surface 30 for transmitting and receiving the ultrasound waves.

In a case where the ultrasound waves are transmitted from the ultrasound transducer 23 to an object, the transmitted ultrasound waves are sequentially reflected from an acoustic impedance discontinuous surface of the object. The ultrasound transducer 23 receives the reflected waves and converts the reflected waves into an electric signal (that is, a reflected wave signal). The reflected wave signal is transmitted to an ultrasound diagnostic apparatus (not shown) at any time. The ultrasound diagnostic apparatus generates the ultrasound tomographic image 120 (see FIG. 3) of the object based on the reflected wave signal output from the ultrasound probe 10.

The ultrasound transducer 23 is accommodated in the case 20. As described above and shown in FIGS. 2 and 4, the case 20 has a substantially cylindrical shape. In the present example, the case 20 has a first case piece 20a and a second case piece 20b. The first case piece 20a is a substantially cylindrical member in which a portion of a peripheral surface is chipped. A lens hole 21 (see FIG. 5) to which the acoustic lens 26 is fitted and an assembly hole 22 (see FIG. 5) to which the second case piece 20b is fitted are formed in the peripheral surface of the first case piece 20a. The assembly hole 22 is a hole that faces the lens hole 21. In a process of manufacturing the ultrasound probe 10, the ultrasound transducer 23 is disposed in the case 20 through the assembly hole 22. The second case piece 20b is attached to the first case piece 20a after the ultrasound transducer 23 and the like are assembled.

However, the configuration of the case 20 is an example and may be appropriately changed. Therefore, the case 20 may be configured by one component or may be configured by three or more components. In addition, in the present example, the acoustic lens 26 is exposed to the outside through the lens hole 21. However, the acoustic lens 26 may be completely accommodated in the case 20. In this case, the lens hole 21 is not formed in the case 20, and the case 20 is made of a material having ultrasound wave transmittance. In addition, in this case, a portion of the case 20 that faces the acoustic lens 26 is the radiation surface 30.

In the case 20, a range bar 44 is provided in a portion that is close to the radiation surface 30 of the ultrasound waves in a circumferential direction. As shown in FIG. 4, a total of two range bars 44 are provided on both sides of the radiation surface 30 in the circumferential direction, respectively. The range bar 44 is a strip-shaped pattern that is elongated in the front-rear direction. The range of the range bar 44 in the front-rear direction is matched with the range of a radiation range Ae of the ultrasound waves in the front-rear direction (see FIG. 3). The range bar 44 is formed by, for example, painting, plating, laser printing, or the like. The range bar 44 functions as a range marker 42 indicating the radiation range Ae of the ultrasound waves. The reason for providing the range marker 42 will be described below.

Further, a scale 38 is provided on the peripheral surface of the case 20. As shown in FIG. 3, the scale 38 has a center mark 40 and a plurality of lines 41 that are arranged at equal intervals from the center mark 40. The center mark 40 is a mark that indicates the center of the radiation range Ae of the ultrasound waves in the axial direction. In the present example, the center mark 40 is an isosceles triangle that faces downward (that is, faces the radiation surface 30). The plurality of lines 41 are arranged at equal intervals on both sides of the center mark 40 in the front-rear direction. Each line 41 extends in the circumferential direction from the inside of the range bar 44 to the outside of the range bar 44. Therefore, it can be said that a portion of the scale 38 overlaps the range marker 42.

In the case 20, a recessed portion 32 that is recessed from a surrounding area is formed on a side that is 180 degrees opposite to the radiation surface 30. A direction plate 36 is disposed in the recessed portion 32. The direction plate 36 is a flat plate having a flat upper surface. A surface of the direction plate 36 is orthogonal to the radiation direction D of the ultrasound waves and functions as the direction marker 34 indicating the radiation direction D of the ultrasound waves, which will be described below.

The case 20 is further provided with two puncture guides 50 and 62 (see FIGS. 2 and 5). Both of the puncture guides 50 and 62 are portions that guide a traveling direction of a puncture needle (not shown). The first puncture guide 50 is disposed closer to a proximal side than the recessed portion 32. The first puncture guide 50 includes a guide hole 52 that penetrates the case 20 in the up-down direction and a lateral hole 58 (see FIG. 2) that connects the guide hole 52 and a side surface of the case 20. As shown in FIG. 5, the guide hole 52 has an hourglass shape whose dimension in the front-rear direction decreases toward the center in the up-down direction. In other words, the guide hole 52 is roughly divided into an upper tapered portion 52a, whose dimension in the front-rear direction decreases as the upper tapered portion 52a becomes further away from an entrance, and a lower tapered portion 52b, whose dimension in the front-rear direction decreases as the lower tapered portion 52b becomes further away from an exit. Hereinafter, one end surface of the upper tapered portion 52a in the front-rear direction is referred to as a “first wall 54”, and the other end surface thereof in the front-rear direction is referred to as a “second wall 56”. In a case where the operator inserts the puncture needle, the operator slides the puncture needle in a state of pressing the puncture needle against the first wall 54 or the second wall 56. Then, the puncture needle advances stably in the direction defined by the first wall 54 or the second wall 56.

A guide marker 60 (see FIGS. 2 and 3) is provided on a side of the case 20 that is opposite to the lateral hole 58. The guide marker 60 is a pattern that indicates a guide direction of the puncture needle by the first puncture guide 50. In the present example, the guide marker 60 is a triangle that is surrounded by a first line indicating the inclination of the first wall 54, a second line indicating the inclination of the second wall 56, and a third line connecting the first line and the second line.

The second puncture guide 62 is disposed at a terminal of the case 20. In the present example, the second puncture guide 62 includes a guide groove 64 (see FIGS. 2 and 5) that is formed in a terminal surface of the case 20. As shown in FIG. 5, the guide groove 64 has a third wall 66 that advances to the proximal side as it goes down. In a case where the operator inserts the puncture needle, the operator slides the puncture needle in a state of pressing the puncture needle against the third wall 66. Then, the puncture needle advances stably in the direction defined by the third wall 66.

Meanwhile, as is clear from the above description, in the present example, the direction plate 36 is provided in the case 20. The reason for providing the direction plate 36 will be described. As described above, the ultrasound probe 10 according to the present example is inserted into the abdominal cavity 112 and is then used. In this case, the position and posture of the ultrasound probe 10 are checked by the camera of the endoscope 100. In addition, the operator knows the internal state of the organ from the ultrasound tomographic image 120 obtained by the ultrasound probe 10.

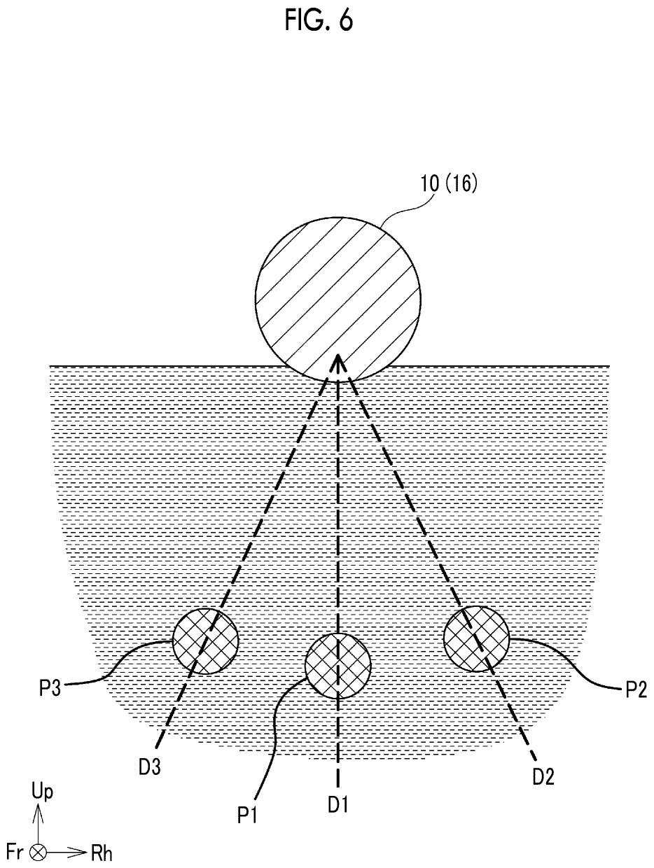

Here, a case where a predetermined treatment is performed on a target part 122 shown in the ultrasound tomographic image 120 (see FIG. 3) is considered. In this case, the operator estimates the actual position of the target part 122 from the position of the target part 122 in the ultrasound tomographic image 120. In a case where the actual position of the target part 122 is estimated, it is necessary to accurately know the radiation direction D of the ultrasound waves. This will be described with reference to FIG. 6. FIG. 6 is a schematic view showing a relationship between the radiation direction D of the ultrasound waves and the position of the target part 122.

For example, a case where the ultrasound tomographic image 120 including the target part 122 is obtained is considered. In a case where the radiation direction D of the ultrasound waves is a direction D1 in FIG. 6, it can be estimated that the target part 122 is located at a position P1. Similarly, in a case where the radiation direction D of the ultrasound waves is a direction D2 in FIG. 6, it can be estimated that the target part 122 is located at a position P2. In a case where the radiation direction D of the ultrasound waves is a direction D3 in FIG. 6, it can be estimated that the target part 122 is located at a position P3. As described above, the radiation direction D of the ultrasound waves is very important in estimating the actual position of the target part 122.

Here, the ultrasound probe according to the related art has a substantially cylindrical shape like the ultrasound probe 10 according to the present example, but is different from the ultrasound probe 10 according to the present example in that it is not provided with a characteristic indicating the radiation direction D of the ultrasound waves. Therefore, in the case of the ultrasound probe according to the related art, it is difficult to know a rotation angle of the case 20 about an axis. As a result, in the case of the ultrasound probe according to the related art, it is difficult for the operator to know the posture of the ultrasound transducer 23 and thus the radiation direction D of the ultrasound waves and to accurately estimate the actual position of the target part 122.

In contrast, as described above, in the ultrasound probe 10 according to the present example, the direction plate 36 is provided in the case 20. An upper surface of the direction plate 36 is orthogonal to the radiation direction D of the ultrasound waves and functions as the direction marker 34 indicating the radiation direction D of the ultrasound waves. Therefore, the operator can observe the direction plate 36 to know the radiation direction D of the ultrasound waves and thus the actual position of the target part 122. In addition, the direction plate 36 is a rectangular shape that is elongated in the front-rear direction. In a case where the direction plate 36 has a simple geometric shape, such as a rectangle, the operator can easily recognize the inclination of the direction plate 36 and thus the radiation direction D of the ultrasound waves from the appearance of the angle of the side or corner portion of the direction plate 36. In addition, since the operator can recognize the radiation direction D of the ultrasound waves, the operator can clearly recognize a positional relationship between the image shown in the ultrasound tomographic image 120 and the actual organ and easily and accurately estimate the actual position of the target part 122.

Further, in the present example, since the plane functioning as the direction marker 34 is recessed from the surrounding area, the contact of the direction marker 34 with other members is effectively prevented. However, the plane functioning as the direction marker 34 may protrude from the surrounding area in a case where there is no problem with the plane getting caught in a surrounding tissue and the like.

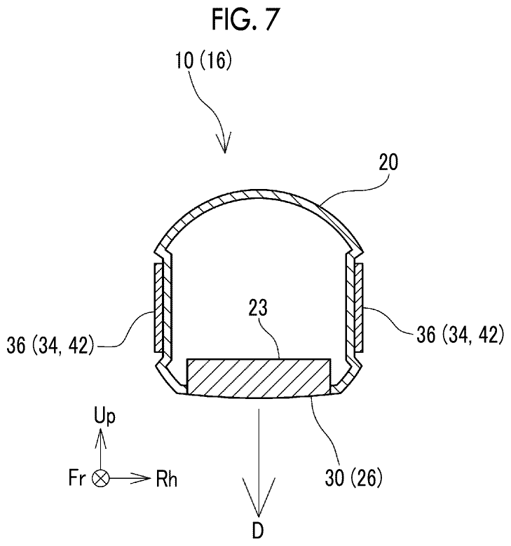

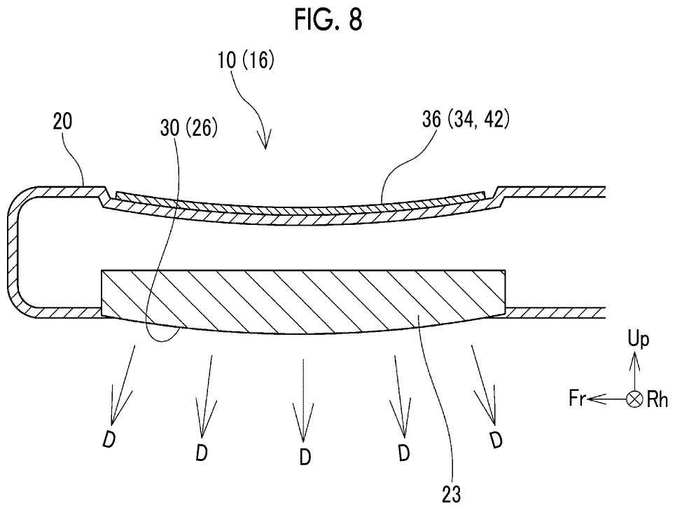

In addition, in the present example, the plane orthogonal to the radiation direction D of the ultrasound waves is provided as the direction marker 34. However, the direction marker 34 may have other forms as long as the radiation direction D of the ultrasound waves can be known. For example, as shown in FIG. 7, a plane parallel to the radiation direction D of the ultrasound waves may be provided as the direction marker 34 in the case 20. In addition, in a case where the ultrasound waves are spread and radiated in a fan shape as in a convex probe and as shown in FIG. 8, the direction marker 34 may be a curved surface that is offset from the curvature of the radiation surface 30. Further, in the example shown in FIG. 8, the surface serving as the direction marker 34 is curved in the axial direction of the case 20. However, the surface serving as the direction marker 34 may be curved about the axis as long as the surface is orthogonal or parallel to the radiation direction D of the ultrasound waves. Furthermore, the number of direction markers 34 is not limited to one, and a plurality of direction markers 34 may be provided. For example, as shown in FIG. 7, two direction markers 34 may be provided at intervals in the circumferential direction of the case 20.

In addition, in order to accurately specify the actual position of the target part 122 shown in the ultrasound tomographic image 120, it is necessary to accurately know not only the radiation direction D of the ultrasound waves but also the radiation range Ae of the ultrasound waves. For example, in a case where the operator resects the target part 122, the operator specifies a distance from an end portion of the ultrasound tomographic image 120 to the target part 122 as a resection margin Mc. The resection margin Mc corresponds to the actual distance from an end portion of the radiation range Ae of the ultrasound waves to the target part 122. Therefore, in a case where the end portion of the radiation range Ae of the ultrasound waves can be specified, the operator can know the actual position of the target part 122.

However, in the ultrasound probe according to the related art, the radiation range Ae of the ultrasound waves is not clearly specified. Therefore, in a case where the ultrasound probe according to the related art is used, it is difficult for the operator to clearly know the end portion of the radiation range Ae. In addition, the ultrasound waves are radiated from the acoustic lens 26. However, since the acoustic lens 26 is pressed against the surface of the organ, most of the acoustic lens 26 is hidden and not seen. In addition, even in a case where the acoustic lens 26 is seen, it is not possible to clearly know the end portion of the radiation range Ae through the observation of the acoustic lens 26 since the acoustic lens 26 is slightly larger than the radiation range Ae. Therefore, the operator is not able to clearly know the actual position of the target part 122 because the operator is not able to clearly know the end portion of the radiation range Ae.

In contrast, in the present example, as described above, the range bar 44 indicating the radiation range Ae is provided in the case 20. The range bar 44 is provided on both sides of the radiation surface 30 in the circumferential direction. Therefore, even in a state in which the radiation surface 30 is pressed against the surface of the organ, the operator can easily visually recognize the range bar 44. As a result, the operator can easily know the end portion of the radiation range Ae and the actual position of the target part 122. In addition, since the range bar 44 is provided on both sides of the radiation surface 30, the operator can clearly know the radiation range Ae regardless of whether the endoscope 100 is on the left or right side of the ultrasound probe 10.

Meanwhile, in order to appropriately treat the target part 122, a mark indicating the end portion of the radiation range Ae may be placed on the surface of the organ. The marking is performed, for example, by burning a very small portion of the surface of the organ. In a case where the range bar 44 is far away from the surface of the organ, a marking position is likely to deviate. In the present example, the range bar 44 is disposed close to the radiation surface 30. Therefore, in a case where the radiation surface 30 is pressed against the surface of the organ, the range bar 44 is naturally close to the surface of the organ. Therefore, this enables the operator to accurately mark the end portion of the radiation range Ae on the surface of the organ.

In addition, as described above, the scale 38 is disposed to partially overlap the range bar 44. Therefore, the operator can clearly know the distance as well as the radiation range Ae. Therefore, this enables the operator to clearly know the actual position of the target part 122.

Meanwhile, it is also considered that the range bar 44 is not provided and the scale 38 is used to know the end portion of the radiation range Ae. However, in general, gradations (intervals) of the scale 38 are set independently of the distance of the radiation range Ae, and the line 41 of the scale 38 deviates from the end portion of the radiation range Ae. That is, the gradation of the scale 38 is set to a readable value, for example, a readable value of 5 mm or 10 mm. In a case where the radiation range Ae is not an integer multiple of the gradation, for example, is 23 mm, the line 41 of the scale 38 deviates from the end portion of the radiation range Ae. Of course, in a case where the gradation of the scale 38 is significantly reduced, for example, in a case where the gradation of the scale 38 is set to 1 mm, it is possible to match the line 41 of the scale 38 with the end portion of the radiation range Ae. However, in this case, since the number of lines 41 of the scale 38 increases, it is difficult for the operator to read the scale 38. That is, it is difficult to represent the end portion of the radiation range Ae with the scale 38 in consideration of the visibility of the scale 38. Therefore, in the present example, the range bar 44 indicating the radiation range Ae is provided separately from the scale 38.

In addition, in the present example, the range of the direction plate 36 in the front-rear direction is matched with the range of the radiation range Ae in the front-rear direction. Therefore, the direction plate 36 functions not only as the direction marker 34 indicating the radiation direction D but also as the range marker 42 indicating the radiation range Ae. As a result, it can be said that the ultrasound probe 10 according to the present example has three range markers 42 in the circumferential direction. The interval at which the three range markers 42 are disposed is less than 180 degrees. Therefore, at least one range marker 42 can be visually recognized from any direction in 360 degrees around the case 20. Therefore, even in a case where only a narrow visual field 102 (see FIG. 1) is obtained as in the endoscope 100, the operator can reliably know the range marker 42 and thus the radiation range Ae. For example, the range bar 44 may not be visible to the camera of the endoscope 100 depending on the positional relationship between the endoscope 100 and the ultrasound probe 10. Even in this case, the operator can see the direction plate 36 to clearly know the radiation range Ae.

Further, in the present example, since the direction plate 36 is also used as the range marker 42, the range of the direction plate 36 in the front-rear direction is matched with the range of the radiation range Ae of the ultrasound waves in the front-rear direction. However, the range of the direction marker 34 in the front-rear direction may not be necessarily matched with the range of the radiation range Ae in the front-rear direction as long as at least a portion thereof overlaps the range of the radiation range Ae of the ultrasound waves in the front-rear direction. An area around the radiation range Ae of the ultrasound waves is an area that the operator pays the most attention to. Since the direction marker 34 is disposed such that at least a portion of the direction marker 34 overlaps the area, the operator can observe the area around the radiation range Ae more carefully because the movement of the line of sight of the operator is reduced.

Further, as described above, in the present example, the guide marker 60 indicating a puncture direction is provided on the peripheral surface of the case 20. The provision of the guide marker 60 enables the operator to easily know the traveling direction of the puncture needle. Therefore, this reduces the need to reinsert the puncture needle and makes it possible to achieve a minimally invasive treatment.

In addition, all of the colors of the range bar 44, the scale 38, the direction plate 36, and the guide marker 60 are opposite to the color of the case 20. For example, in a case where the color of the case 20 is a light color, such as white or silver, the color of the direction marker or the like is a dark color such as black or gray. This coloring makes it possible to improve the visibility of the range bar 44 or the like and makes it possible for the operator to easily recognize the radiation range Ae and the radiation direction D of the ultrasound waves and the guide direction of the puncture needle.

In addition, the configurations described above are only examples, and other configurations may be changed as appropriate as long as the ultrasound probe 10 has the characteristics described in claim 1. Therefore, the number of direction markers 34 and the form and position of the direction marker 34 may be appropriately changed as long as the direction marker 34 indicates the radiation direction D of the ultrasound waves. In addition, the ultrasound probe 10 described above includes the scale 38, the range marker 42, and the guide marker 60 in addition to the direction marker 34. However, as long as the ultrasound probe 10 has the direction marker 34, the other markers may be omitted. Furthermore, the ultrasound probe 10 described above is a linear probe in which the plurality of transducer elements 24 are linearly arranged. However, the technology disclosed in the present specification is not limited to the linear probe and may be applied to other types of probes, for example, convex probes.

In addition, some techniques have been proposed in which the ultrasound tomographic image 120 is displayed to be superimposed on an image (hereinafter, referred to as a “camera image”) captured by the camera of the endoscope 100. In this case, a pattern that functions as an AR marker is provided on the ultrasound probe 10, and the position and posture of the ultrasound probe 10 are specified from the AR marker shown in the camera image. Then, a three-dimensional positional relationship between the camera image and the ultrasound tomographic image 120 is specified from the specified position and posture, and a superimposition position of the ultrasound tomographic image 120 on the camera image and the like is determined. The direction marker 34, the scale 38, the range marker 42, and the guide marker 60 may be used as the AR marker.

What is claimed is:

1. An ultrasound probe inserted into a body cavity, the ultrasound probe comprising:
an ultrasound transducer that radiates an ultrasound wave; and
a case that has a substantially cylindrical shape and that accommodates the ultrasound transducer,
wherein the case includes a direction marker that is a surface indicating a radiation direction of the ultrasound wave, and
the direction marker is provided at a position where the direction marker is visible from an outside and at least a portion of the direction marker overlaps a radiation range of the ultrasound wave in an axial direction.

2. The ultrasound probe according to claim 1,
wherein a range of the direction marker in the axial direction is the same as a range of the radiation range of the ultrasound wave in the axial direction, and
the direction marker also functions as a range marker that indicates the radiation range of the ultrasound wave.

3. The ultrasound probe according to claim 1,
wherein the direction marker is a surface that is parallel to the radiation direction of the ultrasound wave or a surface that is orthogonal to the radiation direction of the ultrasound wave.

4. The ultrasound probe according to claim 3,
wherein the direction marker is a substantially rectangular plane that is provided on a side opposite to a radiation surface of the ultrasound wave in a circumferential direction.

5. The ultrasound probe according to claim 4,
wherein the case has a recessed portion that is recessed from a surrounding area, and
the direction marker is disposed in the recessed portion.

6. The ultrasound probe according to claim 1,
wherein the case further includes one or more range markers that are provided at positions different from a position of the direction marker and that indicate the radiation range of the ultrasound wave.

7. The ultrasound probe according to claim 1,
wherein a color of the direction marker is opposite to a color of the case.
